SEQUENCE LISTING (19 sequences)

SEQ ID NO: 1 - DNA, 45 bp, Artificial
  Synthetic Pro sequence
gggactttccggggaggcgtggcctgggcgggactggggagtggc

SEQ ID NO: 2 - DNA, 23 bp, Artificial
  Synthetic Pro4 sequence
gggactttccggggaggtgtggc

SEQ ID NO: 3 - RNA, 23 nt, Artificial
  Synthetic rPro4 sequence
gggacuuuccggggagguguggc

SEQ ID NO: 4 - DNA, 22 bp, Artificial
  Synthetic Pro3 sequence
agggaggcgtggcctgggcggg

SEQ ID NO: 5 - RNA, 22 nt, Artificial
  Synthetic rPro3 sequence
agggaggcguggccugggcggg

SEQ ID NO: 6 - DNA, 19 bp, Artificial
  Synthetic Pro2 sequence
tggcctgggcgggactggg

SEQ ID NO: 7 - RNA, 19 nt, Artificial
  Synthetic rPro2 sequence
uggccugggcgggacuggg

SEQ ID NO: 8 - DNA, 20 bp, Artificial
  Synthetic Pro1 sequence
gggcgggactggggagtggc

SEQ ID NO: 9 - RNA, 20 nt, Artificial
  Synthetic rPro1 sequence
gggcgggacuggggaguggc

SEQ ID NO: 10 - DNA, 20 bp, Artificial
  Synthetic PPT sequence
aaggggggactggatgggct

SEQ ID NO: 11 - DNA, 33 bp, Artificial
  Synthetic CPPT sequence
aaggggggattggggggtacagtgcagggggaa

SEQ ID NO: 12 - RNA, 33 nt, Artificial
  Synthetic rCPPT sequence
aaggggggauugggggguacagugcagggggaa

SEQ ID NO: 13 - RNA, 20 nt, Artificial
  Synthetic rPPT sequence
aaggggggacuggaugggcu

SEQ ID NO: 14 - DNA, 9 bp, Artificial
  Synthetic L33 sequence
tacagtgca

SEQ ID NO: 15 - RNA, 9 nt, Artificial
  Synthetic rL33 sequence
uacagugca

SEQ ID NO: 16 - DNA, 7 bp, Artificial
  Synthetic linker sequence
actttcc

SEQ ID NO: 17 - RNA, 7 nt, Artificial
  Synthetic linker sequence
acuuucc

SEQ ID NO: 18 - DNA, 7 bp, Artificial
  Synthetic linker sequence
actttcc

SEQ ID NO: 19 - RNA, 7 nt, Artificial
  Synthetic Linker sequence
acuuucc

(19) United States
(12) Patent Application Publication
(10) Pub. No.: US 2017/0191059 A1
(43) Pub. Date: Jul. 6, 2017

(54) NUCLEIC ACIDS ACTING AS DECOYS FOR THE TREATMENT OF LENTIVIRUS INFECTION

(71) Applicants: INSERM (INSTITUT NATIONAL DE LA SANTÉ ET DE LA REC HERCHE MÉDICALE), Paris (FR); CENTRE NATIONAL DE LA RECHERCHE SCIENTIFIQUE (CNRS), Paris (FR); UNIVERSITÉ DE BORDEAUX, Bordeaux (FR)

(72) Inventors: Samir AMRANE, Pessac (FR); Jean-Louis MERGNY, Pessac (FR); Marie-Aline ANDREOLA, Bordeaux (FR); Amina BEDRAT, Pessac (FR)

(21) Appl. No.: 15/313,151

(22) PCT Filed: May 22, 2015
(86) PCT No.: PCT/EP2015/061356

§ 371 Date: Nov. 22, 2016

(30) Foreign Application Priority Data
May 23, 2014 (EP) 14305763.6

(51) Int. Cl. C12N 15/115; C12N 15/113

(52) U.S. Cl. CPC C12N 15/115; C12N 15/1132; C12N 2310/13

(57) ABSTRACT

The present invention relates to nucleic acid sequences which are fragments of HIV-1 genome. The nucleic acids of the present invention are use as decoys for the treatment of lentivirus infection. In particular, the present invention relates to nucleic acids capable of forming at least one G-quadruplex domain and that are capable of inhibiting the replication of at least one lentivirus, such as HIV-1.

### FIELD OF THE INVENTION

The present invention relates to nucleic acid sequences which are fragments of HIV-1 genome. The nucleic acids of the present invention are use as decoys for the treatment of lentivirus infection.

### BACKGROUND OF THE INVENTION

Human immunodeficiency virus (HIV-1) is a retrovirus responsible of a global pandemic inducing a deficiency of the immune system causing AIDS. HIV-1 retrovirus infects cells that carry CD4 and one of the chemokine receptors CCRS or CXCR4¹. After infection, the two HIV-1 single-stranded RNAs are reverse transcribed by the viral reverse transcriptase into double-stranded DNA. The viral DNA is then integrated into the genome of the infected cell. The host cell machinery transcribes the viral genes, new viral proteins are synthesized, and new viruses are finally assembled. At the end of the 1990, the setting of an antiviral therapy targeting different enzymes of the viral cycle was a tremendous step forward in the battle against AIDS. However this treatment did not succeed into the definitive eradication of the virus and due to some mutations in the genome of the virus, resistance against these molecules can occur. Indeed, according to UNAIDS, 34 million people are currently infected by HIV-1. The discovery of new anti-viral strategies is still an important issue.

DNA or RNA sequences containing guanine tracts are able to adopt non-canonical four-stranded structures called G-quadruplexes (G4s)². The core of the G4 is based on the stacking of 2 or more G-tetrads. Each tetrad is a planar association of four guanines held together by eight hydrogen bonds and coordinated with a central Na⁺ or K⁺ cation. Unlike the canonical duplex, G4s form a very polymorphic family of globularly shaped nucleic acid structures. G4s can be thermally stable with melting temperatures typically above 40° C. under near physiological conditions. Genome scale bioinformatics analysis showed a significant enrichment of these sequences in regulatory elements of the human genome such as telomeres and oncogenes. In vivo studies, using specific G4 probes^{3,4} strongly suggests the formation of G4s in cells. The implication of G-quadruplexes in virology⁵ is also the subject of recent investigations in the papilloma, Epstein-Barr and SARS viruses. However, use of G-quadruplexes derived from the own virus genome sequence for the treatment of HIV-1 infection has never been suggested in the prior art

### SUMMARY OF THE INVENTION

The present invention relates to nucleic acid sequences which are fragments of HIV-1 genome. The nucleic acids of the present invention are use as decoys for the treatment of lentivirus infection. In particular, the present invention is defined by the claims.

### DETAILED DESCRIPTION OF THE INVENTION

Several studies show that the viral proteins are able to recognize G4 structures with high affinity and specificity^{6,7}. This striking trend prompted the inventors to search for G4 forming sequences in the HIV-1 genome that could be recognised by the viral proteins in vivo. Using a bioinformatics approach they identified three very conserved G4 forming sequences that are involved in key steps of the HIV-1 replication cycle. The inventors purchased and tested these oligonucleotides in a viral infectivity test and they showed that these sequences are very potent HIV-1 inhibitors with effects in the nanomolar range. These G4s might therefore act as decoys that trap crucial proteins involved in the recognition of the same sequences present in the HIV-1 genome. Accordingly, the present invention relates to nucleic acids capable of forming at least one G-quadruplex domain and that are capable of inhibiting the replication of at least one lentivirus, such as HIV-1.

The term “lentivirus” as used herein, refers to human immunodeficiency virus-1 (HIV-1); human immunodeficiency virus-2 (HIV-2); simian immunodeficiency virus (SIV); feline immunodeficiency virus (FIV) and equine immunodeficiency virus (EIV)

As used herein the term “G-quadruplex domain” refers to any guanosine-rich oligonucleotide sequence capable of forming G-tetrads, each of which is a square arrangement of guanines stabilized by Hoogsteen hydrogen bonding, and which may be further stabilized by the presence of a monovalent cation (especially potassium) in the center of the tetrads, without further limitation as to sequence. A G-quadruplex structure may include 2, 3, 4, 5 or more tetrads. A G-quadruplex structure may be formed of DNA, RNA or a modified nucleic acid such as an LNA or a PNA. Resources including algorithms for identifying and predicting sequences which have the capacity to form G-quadruplexes are readily available, for example online and in QUADRUPLEX NUCLEIC ACIDS, Neidle & Balasubramanian (Eds.) 2006.

In all subsequent paragraphs, all nucleic acid sequences are listed in the 5′ to 3′ direction.

In some embodiments, the present invention relates to a nucleic acid (NA3) having the general formula (I) of:

L3₁-(G)₄₋₆-L3₂-(G)₄₋₆-L3₃-(G)₃₋₄-L3₄ (I)

wherein
(G)ₙ represents a sequence of n guanosines
L3₁ represents a sequence of 0 to 4 nucleotides
L3₂ represents a sequence of 2 to 5 nucleotides
L3₃ represents a sequence of 5 to 10 nucleotides
L3₄ represents a sequence of 0 to 4 nucleotides

As used herein the terms “nucleotide” has its general meaning in the art and includes, but is not limited to, a natural nucleotide, a synthetic nucleotide, or a nucleotide analogue. The nucleoside phosphate may be a nucleoside monophosphate, a nucleoside diphosphate or a nucleoside triphosphate. The sugar moiety in the nucleoside phosphate may be a pentose sugar, such as ribose, and the phosphate esterification site may correspond to the hydroxyl group attached to the C-5 position of the pentose sugar of the nucleoside. A nucleotide may be, but is not limited to, a deoxyribonucleoside triphosphate (dNTP) or a ribonucleoside triphosphate (NTP). The nucleotides may be represented using alphabetical letters (letter designation), as described in Table A. For example, A denotes adenosine (i.e., a nucleotide containing the nucleobase, adenine), C denotes cytosine, G denotes guanosine, and T denotes thymidine. W denotes either A or T/U, and S denotes either G or C. N represents a random nucleotide (i.e., N may be any of A, C, G, or T/U). As used herein, the term “nucleotide analogue” refers to modified compounds that are structurally similar to naturally occurring nucleotides. The nucleotide analogue may have an altered phosphorothioate backbone, sugar moiety, nucleobase, or combinations thereof. Generally, nucleotide analogues with altered nucleobases confer, among other things, different base pairing and base stacking properties. Nucleotide analogues having altered phosphate-sugar backbone (e.g., PNA, LNA, etc.) often modify, among other things, the chain properties such as secondary structure formation. At times in the instant application, the terms “nucleotide analogue,” “nucleotide analogue base,” “modified nucleotide base,” or “modified base” may be used interchangeably.

The term “nucleic acid” refers to a macromolecule composed of chains of monomeric nucleotides, which forms a structure that demonstrates a biological function and may also carry genetic information. As is the case with polynucleotides, nucleic acids encompass DNA and RNA in double- and single-stranded forms, and also encompass nucleic acids wherein the bases are a modified form of either type of nucleotide, including LNAs, PNAs, HNAs, GNAs and TNAs. It will be understood that nucleic acids that comprise a nucleotide sequence as disclosed herein also encompass those nucleic acids wherein thymidine (T) may be replaced in the sequence by uracil (U), such as when uracil (U) in an RNA sequence replaces thymidine (T) in a corresponding DNA sequence.

As used herein, the term “oligonucleotide” means any macromolecule that is a polymer of monomeric nucleotides. The nucleotiode bases may be either ribonucleotides or deoxynucleotides or a modified form of either type of nucleotide, including those that display increased thermal stabilities when hybridized to complementary DNAs or RNAs as compared to unmodified DNA:DNA and DNA:RNA pairs. Such modified nucleotides include morpholino and locked nucleic acids (LNAs), peptide nucleic acids (PNAs), 1′, 5′-anhydrohexitol nucleic acids (HNAs), glycol nucleic acids (GNAs) and threose nucleic acids (TNAs), all of which are characterized by changes to the backbone of the molecule and are capable of folding to form quadruplex structures. The term “polynucleotide” also is meant to encompass single and double stranded forms of nucleotides. Polynucleotides that comprise a nucleotide sequence as disclosed herein also encompass those polynucleotides wherein thymidine (T) may be replaced in the sequence by uracil (U), such as when uracil (U) in an RNA sequence replaces thymidine (T) in a corresponding DNA sequence, inasmuch as one of the four major bases in RNA is uracil (U) rather than thymidine (T) as in DNA.

In some embodiments, L3₁ represents AA.

In some embodiments, L3₂ represents ATT or AUU.

In some embodiments, L3₃ represents TACAGTGCA or UACAGUGCA.

In some embodiments, L3₄ represents AA.

In some embodiments, the nucleic acid (NA3) is represented by SEQ ID NO: 11 or SEQ ID NO:12.

In some embodiments, the present invention relates to a nucleic acid (NA9) having the general formula (II) of:

L9₁-(G)₄₋₆-L9₂-(G)₂₋₃-L9₃-(G)₂₋₄-L9₄ (II)

Wherein
(G)ₙ represents a sequence of n guanosines
L9₁ represents a sequence of 0 to 4 nucleotides
L9₂ represents a sequence of 2 to 4 nucleotides
L9₃ represents a sequence of 1 to 4 nucleotides
L9₄ represents a sequence of 0 to 4

In some embodiments, L9₁ represents AA.

In some embodiments, L9₂ represents ACT or ACU.

In some embodiments, L9₃ represents AT or AU.

In some embodiments, L9₄ represents AA.

In some embodiments, the nucleic acid (NA9) is represented by SEQ ID NO:10 or SEQ ID NO:13.

In some embodiments, the present invention relates to a nucleic acid (NA10) having the general formula (III) of:

L10₀-(G)₃₋₄-L10₁-(G)₃₋₄-L10₂-(G)₂₋₃-L10₃-(G)₂₋₃-L10₄-(G)₃₋₄-L10₅-(G)₃₋₄-L10₆-(G)₃₋₄-L10₇-(G)₂₋₃-L10₈ (III)

Wherein
(G)ₙ represents a sequence of n guanosines
L10₀ represents a sequence of 0 to 4 nucleotides
L10₁ represents a sequence of 7 to 10 nucleotides
L10₂ represents a sequence of 1 to 3 nucleotides
L10₃ represents a sequence of 1 to 4 nucleotides
L10₄ represents a sequence of 2 to 4 nucleotides
L10₅ represents a single nucleotide
L10₆ represents a sequence of 2 to 5 nucleotides
L10₇ represents a sequence of 2 to 5 nucleotides
L10₈ represents a sequence of 0 to 4 nucleotides

In some embodiments, L10₁ represents ACTTTCC or ACUUUCC.

In some embodiments, L10₂ represents A.

In some embodiments, L10₃ represents CGT or CGU.

In some embodiments, L10₄ represents CCT or CCU.

In some embodiments, L10₅ represents C.

In some embodiments, L10₆ represents ACT or ACU.

In some embodiments, L10₇ represents AGT or AGU.

In some embodiments, the nucleic acid (NA10) is represented by SEQ ID NO:1 (GGGACTTTCCGGGGAGGCGTGGCCTGGGCGGGACTGGGGAGTGGC).

In some embodiments, the present invention relates to a nucleic acid (NA10.1) having the general formula (IV) of:

L10₀-(G)₃₋₄-L10₁-(G)₃₋₄-L10₂-(G)₂₋₃-L10₃-(G)₂₋₃-L10₄ (IV)

Wherein
(G)ₙ represents a sequence of n guanosines
L10₀ represents a sequence of 0 to 4 nucleotides
L10₁ represents a sequence of 7 to 10 nucleotides
L10₂ represents a sequence of 1 to 3 nucleotides
L10₃ represents a sequence of 1 to 4 nucleotides
L10₄ represents a sequence of 0 to 4 nucleotides

In some embodiments, L10₁ represents ACTTTCC or ACUUUCC.

In some embodiments, L10₂ represents A.

In some embodiments, L10₃ represents CGT or CGU.

In some embodiments, L10₄ represents C.

In some embodiments, the nucleic acid (NA10.1) is represented by SEQ ID NO:2 or SEQ ID NO:3.

In some embodiments, the present invention relates to a nucleic acid (NA10.2) having the general formula (V) of:

L10₁-(G)₃₋₄-L10₂-(G)₂₋₃-L10₃-(G)₂₋₃-L10₄-(G)₃₋₄-L10₅-(G)₃₋₄-L10₆ (V)

Wherein
L10₁ represents a sequence of 0 to 4 nucleotides
L10₂ represents a sequence of 1 to 3 nucleotides
L10₃ represents a sequence of 1 to 4 nucleotides
L10₄ represents a sequence of 2 to 4 nucleotides
L10₅ represents a single nucleotide
L10₆ represents a sequence of 0 to 4 nucleotides

In some embodiments, L10₁ represents A.

In some embodiments, L10₂ represents A.

In some embodiments, L10₃ represents CGT or CGU.

In some embodiments, L10₄ represents CCT or CCU.

In some embodiments, L10₅ represents C.

In some embodiments, the nucleic acid (NA10.2) is represented by SEQ ID NO:4 or SEQ ID NO:5.

In some embodiments, the present invention relates to a nucleic acid (NA10.3) having the general formula (VI) of:

L10₃-(G)₂₋₃-L10₄-(G)₃₋₄-L10₅-(G)₃₋₄-L10₆-(G)₃₋₄-L10₇ (VI)

Wherein
(G)ₙ represents sequence of n guanosines
L10₃ represents a sequence of 0 to 4 nucleotides
L10₄ represents a sequence of 2 to 4 nucleotides
L10₅ represents a single nucleotide
L10₆ represents a sequence of 2 to 5 nucleotides
L10₇ represents a sequence of 0 to 4 nucleotides

In some embodiments, L10₃ represents T or U.

In some embodiments, L10₄ represents CCT or CCU.

In some embodiments, L10₅ represents C.

In some embodiments, L10₆ represents ACT or ACU.

In some embodiments, the nucleic acid (N10.3) is represented by SEQ ID NO:6 or SEQ ID NO:7.

In some embodiments, the present invention relates to a nucleic acid (NA10.4) having the general formula (VII) of:

L10₄-(G)₃₋₄-L10₅-(G)₃₋₄-L10₆-(G)₃₋₄-L10₇-(G)₂₋₃-L10₈ (VII)

Wherein
(G)ₙ represents a sequence of n guanosines
L10₄ represents a sequence of 0 to 4 nucleotides
L10₅ represents a single nucleotide
L10₆ represents a sequence of 2 to 5 nucleotides
L10₇ represents a sequence of 2 to 5 nucleotides
L10₈ represents a sequence of 0 to 4 nucleotides

In some embodiments, L10₄ represents CCT or CCU.

In some embodiments, L10₅ represents C.

In some embodiments, L10₆ represents ACT or ACU.

In some embodiments, L10₇ represents AGT or AGU.

In some embodiments, the nucleic acid (NA10.4) is represented by SEQ ID NO:8 or SEQ ID NO:9.

For use in the instant invention, the nucleic acids of the present invention can be synthesized de novo using any of a number of procedures well known in the art. Chemical synthesis can be performed by a variety of automated nucleic acid synthesizers available in the market. These nucleic acids may be referred to as synthetic nucleic acids. Alternatively, the nucleic acids of the present invention can be produced on a large scale in plasmids. The nucleic acids of the present invention can be prepared from existing nucleic acid sequences using known techniques, such as those employing restriction enzymes, exonucleases or endonucleases.

A further object of the present invention relates to a method of treating a lentivirus infection in a subject in need thereof comprising administering the subject with a therapeutically effective amount of at least one nucleic acid of the present invention.

As used herein, the term “subject” denotes a mammal, such as a rodent, a feline, a canine, a equine and a primate. Preferably, a subject according to the invention is a human.

In some embodiments, the method of the present invention is particularly suitable for the treatment of HIV-1 infections.

By a “therapeutically effective amount” is meant a sufficient amount of nucleic acid of the present invention to treat and/or to prevent lentivirus infections (e.g. HIV-1 infections) at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The nucleic acids of the present invention can be administered by known routes of administration including intravenous administration, intramuscular, intraperitoneal, intracerebrospinal, subcutaneous, intra-articular, intrasynovial, intrathecal, oral, topical, or inhalation routes. Effective dosages and schedules for administering antagonists or agonists are determined empirically according to guidelines generally recognized by those of skill in the art. Single or multiple dosages may be employed.

As noted above, the nucleic acids of the present invention useful in the methods of the present disclosure can be incorporated into pharmaceutical compositions suitable for administration into an animal such as a mammal. Methods for formulating such compositions are generally well known. Guidance is available for example from Remington: THE SCIENCE AND PRACTICE OF PHARMACY, 19th Edition, Gennaro (ed.) 1995, Mack Publishing Company, Easton, Pa. Such compositions typically comprise at least one anti-RT aptamer and a pharmaceutically acceptable carrier. The term “pharmaceutically acceptable carrier” refers to any and all coatings, excipients, solvents, dispersion media, absorption delaying agents, and the like, compatible with pharmaceutical administration. Such carriers also include for example sodium chloride, colloidal silica, talc, various polymeric carriers including polyvinyl pyrrolidone, cellulose-based compounds such as carboxymethylcellulose or methylcellulose, polyvinylpyrrolidone, polyacrylates, and polyethylene glycol. Dosage forms include, for example, oral or sublingual tablets, pellets, micro- and nano-capsules, liposomes, inhalation forms, nasal sprays, and sustained-release preparations. Solutions or suspensions used for administering nucleic acids of the present invention can include one or more of the following components: a sterile diluent such as water for injection, saline solution; fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose. In some embodiments, a pharmaceutical composition can be delivered via slow release formulation or matrix comprising nucleic acids of the present invention or DNA constructs suitable for expression of nucleic acids of the present invention in or around a site within the body.

In some embodiments, the nucleic acid of the present invention of the invention may be formulated into pharmaceutical compositions that can be used to apply microbicides to effectively prevent transmission of HIV-1 through mucosae, more particularly to prevent the sexual or vaginal transmission of HIV-1. Thus, the compositions are in forms adapted to be applied to the site where sexual intercourse or related intimate contact takes place, such as the genitals, vagina, vulva, cervix, rectum, mouth, hands, lower abdomen, upper thighs, especially the vagina, vulva, cervix, and ano-rectal mucosae. As appropriate topical compositions there may be cited for example gels, jellies, creams, pastes, emulsions, dispersions, ointments, films, sponges, foams, aerosols, powders, intravaginal rings or other intravaginal drug delivery systems, cervical caps, implants, patches, suppositories or pessaries for rectal, or vaginal application, vaginal or rectal or buccal tablets, mouthwashes. The present topical formulations such as the gel formulations described herein could, for example, be applied into the vagina by hand, suppositories, or conventional tampon or syringe techniques. The method of administering or delivering the gel into the vagina is not critical so long as an effective amount of the gel is delivered into the vagina. The present topical formulations such as the gel formulations described herein may also be used for protection during anal intercourse and can be applied using similar techniques. For vaginal heterosexual intercourse, the present topical formulations such as the gel formulations described herein may be applied into the vagina prior to intercourse. For anal intercourse (heterosexual or homosexual), the present topical formulations such as the gel formulations described herein may be inserted into the rectum prior to intercourse. For either vaginal or anal intercourse, the present topical formulations such as the gel formulations described herein may also act as a lubricant. For added protection it is generally preferred that the present topical formulations such as the gel formulations described herein be applied before intercourse or other sexual activity and that, if appropriate, a condom be used. For even further protection, the present topical formulations such as the gel formulations described herein can be applied as soon as possible after completion of the sexual activity. Although application only after the sexual activity is less recommended, it would still be desirable afterwards if the application was not performed prior to the sexual activity for any reason (e.g., in cases of rape).

In some embodiments, the nucleic acid of the present inventions of the invention may be used in all the suitable formulations, alone or in combination with other active ingredients, such as antivirals, antibiotics, immunomodulators or vaccines. They may also be used alone or in combination with other prophylactic agents for the prevention of viral infections. Thus, the nucleic acid of the present inventions of the invention may be combined with pharmaceutically acceptable adjuvants conventionally employed in vaccines and administered in prophylactically effective amounts to protect individuals over an extended period of time against HIV-1 infection. Antiviral compounds which may be used in combination with the nucleic acid of the present inventions of the invention may be known antiretroviral compounds such as pentamidine, thymopentin, castanospermine, dextran (dextran sulfate), foscarnet-sodium (trisodium phosphono formate); nucleoside reverse transcriptase inhibitors, e.g. zidovudine (3′-azido-3′-deoxythymidine, AZT), didanosine (2′,3′-dideoxyinosine; ddI), zalcitabine (dideoxycytidine, ddC) or lamivudine (2′-3′-dideoxy-3′-thiacytidine, 3TC), stavudine (2′,3′-didehydro-3′-deoxythymidine, d4T), abacavir and the like; non-nucleoside reverse transcriptase inhibitors such as nevirapine (11-cyclopropyl-5,11-dihydro-4-methyl-6H-dipyrido-[3,2-b:2′,3′-e][1,4]diazepin-6-one), efavirenz, delavirdine, and the like; phosphonate reverse transcriptase inhibitors, e.g. tenofovir and the like; compounds of the TIBO (tetrahydro-imidazo[4,5,1-jk][1,4]-benzodiazepine-2 (1H)-one and thione)-type e.g. (S)-8-chloro-4,5,6,7-tetrahydro-5-methyl-6-(3-methyl-2-butenyl)imidazo-[4,5,1-j k][1,4]benzo-diazepine-2(1H)-thione; compounds of the [alpha]-APA ([alpha]-anilino phenyl acetamide) type e.g. [alpha]-[(2-nitrophenyl)amino]-2,6-dichlorobenzene-acetamide and the like; inhibitors of trans-activating proteins, such as TAT-inhibitors, e.g. RO-5-3335, or REV inhibitors, and the like; protease inhibitors e.g. indinavir, ritonavir, saquinavir, lopinavir (ABT-378), nelfinavir, amprenavir, TMC-126, BMS-232632, VX-175 and the like; fusion inhibitors, e.g. T-20, T-1249 and the like; CXCR4 receptor antagonists, e.g. AMD-3100 and the like; inhibitors of the viral integrase; ribonucleotide reductase inhibitors, e.g. hydroxyurea and the like. Combinations may as well exert a synergistic effect in inhibiting HIV-1 replication when components of the combination act on different or same sites of HIV-1 replication, preferably on different sites. The use of such combinations may reduce the dosage of a given conventional antiretroviral agent which would be required for a desired prophylactic effect as compared to when that agent is administered as a single active ingredient. These combinations reduce potential of resistance to single agent, while minimizing any associated toxicity. These combinations may also increase the efficacy of the conventional agent without increasing the associated toxicity.

The invention will be further illustrated by the following figures and examples. However, these examples and figures should not be interpreted in any way as limiting the scope of the present invention.

### FIGURES

FIG. 1: A. genetic structure of the HIV-1 genome. B. Bioinformatic search of G4 forming sequences. This graphical representation show the score in function of the oligonucleotidic sequence of the HIV genome.

FIG. 2: Examples of UV-melting profiles recorded at 295 nm at a strand concentration of 5 μM for CPPT (ID=11). The experiments were performed in a buffer composed of 20 mM potassium phosphate pH 6.9 supplemented with 70 mM KCl.

FIG. 3: HeLa P4 cells were infected by viral supernatant HIV-f as described in the Methods section. To assess the inhibitory effect of the decoys, HeLa P4 cells were infected with HIV-1 in the presence of various concentrations of oligonucleotide. Viral replication was monitored by Beta-galactosidase activity at 24 h post-infection. Values were normalized to 100% corresponding to Beta-galactosidase activity in the absence of decoys. A, B, C HIV-1 replication percentage measured in the presence of increasing concentrations of decoys from 0.1 nM to 500 nM of decoys. The data were collected for the following sequences: A) PRO1 (ID=8, black squares), PRO2 (ID=6, black circles), PRO3 (ID=4, grey squares), PRO4 (ID=2, black lozenge). B) CPPT (ID=11, black squares), PPT (ID=10, black circles), 93del (grey squares), C) rPRO1 (ID=9, black squares), rPPT (ID=13, black circles), rCPPT (ID=12, grey squares).

### EXAMPLE

Material & Methods

Bioinformatics Analysis:

The 1870 HIV-1 sequences were obtained from the HIV-1 database which provides premade alignments to the community (www.hiv.lanl.gov). The alignment apparently presents around 11 000 nucleotides instead of the usual 9200 nucleotides for the HIV-1 consensus sequence. This is due to the insertion of gaps to optimise the alignment of the 1870 sequences. The score algorithm that we developed in the laboratory (Bedrat et al, in preparation) searches for G/C skewness and the presence of GC blocks in the alignment of the 1870 HIV-1 sequences retrieved from the HIV-1 database. It analyses the genome using a sliding window of 25 nucleotides and attribute a score to the first nucleotide of the window. The average of the 1870 scores obtained for each window is depicted in a graphical representation. We consider that the scores higher than 1 in absolute value (−1 or +1) are potentially able to form DNA or RNA G4 structures in the (+) strand (for positive values) or in the (−) strand for the negative values. The analysis of sequence conservation was performed using the webLOGO software to generate the LOGO representation.

Preparation of the Oligonucleotides:

Oligonucleotides were purchased from Eurogentec (Seraing, Belgium) without further purification (Reverse-Phase Cartridge Gold). Concentrations were determined by ultraviolet (UV) absorption using the extinction coefficients provided by the manufacturer. All oligonucleotides were dissolved in 20 mM potassium phosphate buffer containing 70 mM KCl.

UV-Melting:

UV-melting measurements⁸ were performed on a Uvikon XL (Secomam) spectrophotometer coupled to a water bath temperature-control accessory. A temperature-increase rate of 0.2° C./min was applied and the absorbance values were measured every 1° C. The temperature was measured with an inert glass sensor immersed into a control quartz cell filled with water. The absorbance was monitored at 240 and 295 nm using quartz cells of 0.2 or 1 cm pathlength and 580 μl of volume.

Cell Lines and Viruses:

HeLa P4 cells encoding a Tat-inducible β-galactosidase were maintained in DMEM medium (Invitrogen) supplemented with 10% inactivated FCS, 1 mg/ml geneticin (G418, Gibco-BRL), gentamycin. MT4 and H9Laï cells were grown in RPMI 1640 glutamax medium (Invitrogen) supplemented with 10% inactivated FCS. HIV-1 viruses were obtained after 48 h co-culture of MT4 cells (0.5×10⁶/ml) and H9Laï cells (1×10⁶/ml), chronically infected by HIV-1Laï isolate, in RPMI 1640 glutamax medium supplemented with 10% inactivated FCS, at 37° C. under humidified atmosphere and 5% CO2. The culture was then centrifuged and the supernatant was clarified by filtration on a 0.45 μm membrane before freezing at −80° C.

Viral Infectivity:

The oligonucleotides were preincubated in a 100 mM potassium solution to favour G4 formation. When added they are incubated in presence of the HelaP4 cells 20 minutes before infection. The infectivity was assayed on HeLa P4 cells expressing CD4 receptor and the β-galactosidase gene under the control of the HIV-1 LTR. HeLa P4 were plated using 200 μl of DMEM medium supplemented with 10% inactivated FCS in 96-multi-well plates at 10 000 cells per well. After overnight incubation at 37° C., under humidified atmosphere and 5% CO2, the supernatant was discarded and 200 μl of viral preparation were added in serial dilutions. After 24 h of infection, the supernatant was discarded and the wells were washed 3 times with 200 μl of PBS. Each well was refilled with 200 μl of a reaction buffer containing 50 mM Tris-HCl pH 8.5, 100 mM β-mercaptoethanol, 0.05% Triton X-100 and 5 mM 4-methylumbelliferyl-B-D-galactopyranoside (4-MUG) (Sigma). After 24 h, the reaction was measured in a fluorescence microplate reader (Cytofluor II, Applied Biosystems) at 360/460 nm Ex/Em.

Results

Bioinformatics Search of G4 Forming Sequences in the HIV-1 Genome:

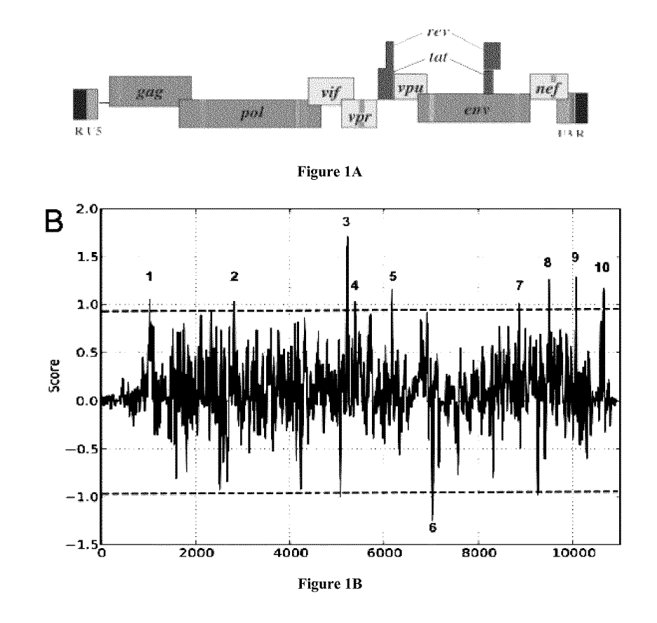

Using our bioinformatic algorithm we searched for sequences that are potentially able to form G4 structures in the HIV-1 genome. We analysed an alignment of 1870 viral sequences provided by the HIV-1 database. We found 10 different loci that could potentially form G4 structures (FIG. 1). Nine of them are present in the (+) strand with scores higher than +1 and one is present in the (−) strand with a score <−1.

Verification of the G4 Formation In Vitro:

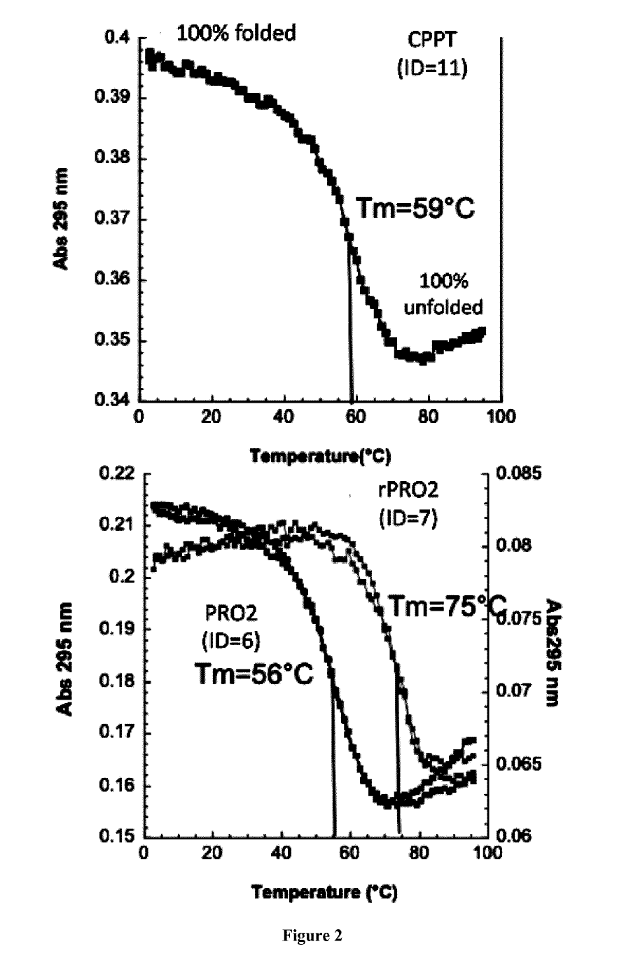

To verify the formation of G4 structures, we purchased the DNA oligonucleotide corresponding to these sequences and we performed UV-melting experiments followed at 295 nm (FIG. 2). At this wavelength the denaturation of the structure generates an hypochromism that is specific of the G4 structures. Amongst the 10 detected candidates, sequence #1, 2 and 4 formed very unstable G4 structures, with melting temperature below 10° C. (data not shown), that might not be compatible with in vivo formation. The seven remaining sequences formed stable RNA and DNA G4 structures with melting temperature ranging from 30° C. to 75° C. In FIG. 2A are presented examples of G4 specific melting profiles. In table 1 are presented the melting temperatures derived from the melting profiles of the 3 most important sequences for this study. We decided to analyse the sequence #10 (45 bases) by synthesizing smaller tracts of 19 to 23 nucleotides spanning the entire sequence. This sequence is therefore able to form 4 different G4 structures (DNA or RNA backbones) with melting temperature ranging from 40° C. to 75° C. We also determined the G4 structure⁹ of the PRO2 sequence (ID=6) by Nuclear Magnetic Resonance spectroscopy. It forms a stable two G-tetrads antiparallel G4 with an additional Watson-Crick CG base pair. We also confirmed that sequence #3 (CPPT (ID=11)) and #9 (PPT (ID=10)) are also able to form stable G4 structures with melting temperature of 59° C. and 35° C.

| | | SEQ | | Tₘ | Inhibition |
|---|---|---|---|---|---|
| No | Name | ID | SEQUENCES | (0° C.) | IC₅₀ (nM) |
| 10 | Pro | 1 | GGGACTTTCCGGGGAGGCGTGGCCTGGGCGGGACTGGGGAGTGGC | — | — |
| 10-1 | Pro4 | 2 | GGGACTTTCCGGGGAGGTGTGGC | 40 | 5.5 |
| | rPro4 | 3 | GGGACUUUCCGGGGAGGUGUGGC (RNA) | 53 | 200 |
| 10-2 | Pro3 | 4 | AGGGAGGCGTGGCCTGGGCGGG | 58 | 45 |
| | rPro3 | 5 | AGGGAGGCGUGGCCUGGGCGGG (RNA) | 70 | <100 |
| 10-3 | Pro2 | 6 | TGGCCTGGGCGGGACTGGG | 56 | 100 |
| | rPro2 | 7 | UGGCCUGGGCGGGACUGGG (RNA) | 75 | ~10 |
| 10-4 | Pro1 | 8 | GGGCGGGACTGGGGAGTGGC | 58 | 1.4 |
| | rPro1 | 9 | GGGCGGGACUGGGGAGUGGC | 69 | 4 |
| 9 | PPT | 10 | AAGGGGGGACTGGATGGGCT | 35 | 6.5 |
| 3 | CPPT | 11 | AAGGGGGGATTGGGGGGTACAGTGCAGGGGGAA | 59 | 0.9 |
| 3 | rCPPT | 12 | AAGGGGGGAUUGGGGGGUACAGUGCAGGGGGAA (RNA) | 65 | 3 |
| 9 | rPPT | 13 | AAGGGGGGACUGGAUGGGCU (RNA) | 45 | 3.5 |

Potential Biological Roles of these Sequences:

According to their locations, we also found that these three sequences might play a role in key steps of the viral cycle: i) Sequence #3 is localised in the center of the viral genome and it contains the C-ppt sequence. This important sequence is the central initiation site of the reverse transcription during the (+) strand DNA synthesis. ii) Sequence #9 is localised at 3′ end of the genome. These sequence is the first initiation site of the reverse transcription during the (+) strand DNA synthesis. iii) Sequence #10 is located on the promoter of the provirus, at 40 nt upstream from the transcription initiation site, close to the TATA box. This sequence overlaps the so-called minimum promoter composed of three SP1 and two NF-kB binding sites which are crucial for the initiation of the transcription of HIV-1. The LOGO representation generated from the alignment of the 1870 sequences shows a high level of conservation of the 3 sequences suggesting an important role involving specific protein recognition of these sequences.

Inhibition of the Viral Infectivity Using a Decoy Strategy:

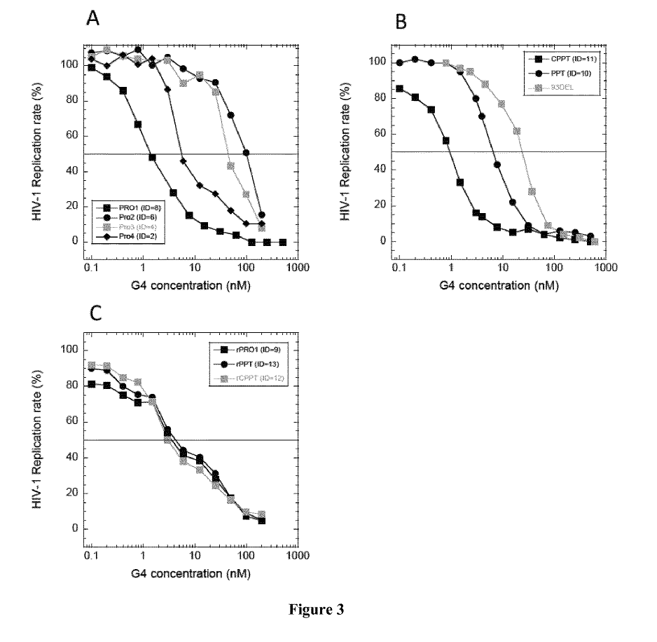

These data prompted us to test if these oligonucleotides are able to inhibit HIV-1 infectivity as already observed for Andevir or Zintevir. We purchased and tested these sequences in a viral infectivity test realised in vivo with real HIV viruses infecting HeLap4 cells. The G4s derived from HIV-1 genome strongly inhibited HIV-1 infectivity with IC₅₀ lower that 4 nM for Sequences #3 and 10-4 (FIG. 3). The inhibition observed for the decoys are much stronger than the ones observed for Andevir (93del) (IC₅₀=25 nM). Cytotoxicity test on HeLaP4 cells performed with the same G4s at a concentration of 500 nM did not reveal any toxicity after 24 hours. These G4s might therefore act as decoys and may trap crucial proteins involved in the recognition of the same sequences present in the HIV-1 genome.

### REFERENCES

Throughout this application, various references describe the state of the art to which this invention pertains. The disclosures of these references are hereby incorporated by reference into the present disclosure.
(1) Pomerantz, R. J.; Horn, D. L. Nat Med 2003, 9, 867.
(2) Webba da Silva, M.; Trajkovski, M.; Sannohe, Y.; Ma'ani Hessari, N.; Sugiyama, H.; Plavec, J. Angew Chem Int Ed Engl 2009, 48, 9167.
(3) Biffi, G.; Di Antonio, M.; Tannahill, D.; Balasubramanian, S. Nat Chem 2014, 6, 75.
(4) Biffi, G.; Tannahill, D.; McCafferty, J.; Balasubramanian, S. Nat Chem 2013, 5, 182.
(5) Murat, P.; Zhong, J.; Lekieffre, L.; Cowieson, N. P.; Clancy, J. L.; Preiss, T.; Balasubramanian, S.; Khanna, R.; Tellam, J. Nat Chem Biol 2014, 10, 358.
(6) Faure-Perraud, A.; Metifiot, M.; Reigadas, S.; Recordon-Pinson, P.; Parissi, V.; Ventura, M.; Andreola, M. L. Antivir Ther 2011, 16, 383.
(7) de Soultrait, V. R.; Lozach, P. Y.; Altmeyer, R.; Tarrago-Litvak, L.; Litvak, S.; Andreola, M. L. J Mol Biol 2002, 324, 195.
(8) Mergny, J. L.; Lacroix, L. Curr Protoc Nucleic Acid Chem 2009, Chapter 17, Unit 171.
(9) Amrane, S.; Kerkour, A.; Bedrat, A.; Vialet, B.; Andreola, M. L.; Mergny, J. L. J Am Chem Soc 2014, 136, 5249.

1. A nucleic acid selected from the group consisting of
i) a nucleic acid (NA3) having the general formula (I) of:
L3₁-(G)₄₋₆-L3₂-(G)₄₋₆-L3₃-(G)₃₋₄-L3₄ (I)
wherein
L3₁ represents a sequence of 0 to 4 nucleotides
L3₂ represents a sequence of 2 to 5 nucleotides
L3₃ represents a sequence of 5 to 10 nucleotides and
L3₄ represents a sequence of 0 to 4 nucleotides
ii) a nucleic acid (NA9) having the general formula (II) of:
L9₁-(G)₄₋₆-L9₂-(G)₂₋₃-L9₃-(G)₂₋₄-L9₄ (II)
wherein
L9₁ represents a sequence of 0 to 4 nucleotides
L9₂ represents a sequence of 2 to 4 nucleotides
L9₃ represents a sequence of 1 to 4 nucleotides and
L9₄ represents a sequence of 0 to 4 nucleotides;
iii) a nucleic acid (NA10) having the general formula (III) of:
L10₀-(G)₃₋₄-L10₁-(G)₃₋₄-L10₂-(G)₂₋₃-L10₃-(G)₂₋₃-L10₄-(G)₃₋₄-L10₅-(G)₃₋₄-L10₆-(G)₃₋₄-L10₇-(G)₂₋₃-L10₈ (III)
wherein
L10₀ represents a sequence of 0 to 4 nucleotides
L10₁ represents a sequence of 7 to 10 nucleotides
L10₂ represents a sequence 1 to 3 nucleotides
L10₃ represents a sequence of 1 to 4 nucleotides
L10₄ represents a sequence 2 to 4 nucleotides
L10₅ represents a single nucleotide
L10₆ represents a sequence of 2 to 5 nucleotides
L10₇ represents a sequence of 2 to 5 nucleotides and
L10₈ represents a sequence of 0 to 4 nucleotides;
iv) a nucleic acid (NA10.1) having the general formula (IV) of:
L10₀-(G)₃₋₄-L10₁-(G)₃₋₄-L10₂-(G)₂₋₃-L10₃-(G)₂₋₃-L10₄ (IV)
wherein
L10₀ represents a sequence of 0 to 4 nucleotides
L10₁ represents a sequence of 7 to 10 nucleotides
L10₂ represents a sequence of 1 to 3 nucleotides
L10₃ represents a sequence of 1 to 4 nucleotides
L10₄ represents a sequence of 0 to 4 nucleotides;
v) a nucleic acid (NA10.2) having the general formula (V) of:
L10₁-(G)₃₋₄-L10₂-(G)₂₋₃-L10₃-(G)₂₋₃-L10₄-(G)₃₋₄-L10₅-(G)₃₋₄-L10₆ (V)
wherein
L10₁ represents a sequence of 0 to 4 nucleotides
L10₂ represents a sequence of 1 to 3 nucleotides
L10₃ represents a sequence of 1 to 4 nucleotides
L10₄ represents a sequence of 2 to 4 nucleotides
L10₅ represents a single nucleotide
L10₆ represents a sequence of 0 to 4 nucleotides;
vi) a nucleic acid (NA10.3) having the general formula (VI) of:
L10₃-(G)₂₋₃-L10₄-(G)₃₋₄-L10₅-(G)₃₋₄-L10₆-(G)₃₋₄-L10₇ (VI)
wherein
L10₃ represents a sequence of 0 to 4 nucleotides
L10₄ represents a sequence 2 to 4 nucleotides
L10₅ represents a single nucleotide
L10₆ represents a sequence of 2 to 5 nucleotides
L10₇ represents a sequence of 0 to 4 nucleotides; and
vii) a nucleic acid (NA10.4) having the general formula (VII) of:
L10₄-(G)₃₋₄-L10₅-(G)₃₋₄-L10₆-(G)₃₋₄-L10₇-(G)₂₋₃-L10₈ (VII)
wherein
L10₄ represents a sequence of 0 to 4 nucleotides
L10₅ represents a single nucleotide
L10₆ represents a sequence of 2 to 5 nucleotides
L10₇ represents a sequence of 2 to 5 nucleotides and
L10₈ represents a sequence of 0 to 4 nucleotides;
and wherein for each of NA3, NA9, NA10, NA10.1, NA10.3, NA10.2, and NA10.3, (G)₂₋₃, (G)₃₋₄ and (G)₄₋₆ represent a sequence of 2 to 3, 3 to 4 or 4 to 6 guanosines, respectively.

2. The nucleic acid of claim 1 wherein the nucleic acid is NA3 and L10₁ represents ACTTTCC or ACUUUCC.

3. The nucleic acid of claim 1 wherein the nucleic acid is NA3 and L10₂ represents A.

4. The nucleic acid of claim 1 wherein the nucleic acid is NA3 and L10₃ represents CGT or CGU.

5. The nucleic acid of claim 1 wherein the nucleic acid is NA3 and L10₄ represents C.

6. The nucleic acid of claim 1 wherein the nucleic acid is NA3 and is represented by SEQ ID NO:2 or SEQ ID NO:3.

7. (canceled)

8. The nucleic acid of claim 1 wherein the nucleic acid is NA9 and L9₁ represents AA.

9. The nucleic acid of claim 1 wherein the nucleic acid is NA9 and L9₂ represents ACT or ACU.

10. The nucleic acid of claim 1 wherein the nucleic acid is NA9 and L9₃ represents AT or AU.

11. The nucleic acid of claim 1 wherein the nucleic acid is NA9 and L9₄ represents AA.

12. The nucleic acid of claim 1 wherein the nucleic acid is NA9 and is represented by SEQ ID NO:10 or SEQ ID NO:13.

13. (canceled)

14. The nucleic acid of claim 1 wherein the nucleic acid is NA10 and L10₁ represents ACTTTCC or ACUUUCC.

15. The nucleic acid of claim 1 wherein the nucleic acid is N109 and L10₂ represents A.

16. The nucleic acid of claim 1 wherein the nucleic acid is NA10 and L10₃ represents CGT or CGU.

17. The nucleic acid of claim 1 wherein the nucleic acid is NA10 and L10₄ represents CCT or CCU.

18. The nucleic acid of claim 1 wherein the nucleic acid is NA10 and L10₅ represents C.

19. The nucleic acid of claim 1 wherein the nucleic acid is NA10 and L10₆ represents ACT or ACU.

20. The nucleic acid of claim 1 wherein the nucleic acid is NA10 and L10₇ represents AGT or AGU.

21. The nucleic acid of claim 1 wherein the nucleic acid is NA10 and is represented by SEQ ID NO:1.

22. (canceled)

23. The nucleic acid of claim 1 wherein the nucleic acid is NA10.1 and L10₁ represents ACTTTCC or ACUUUCC.

24. The nucleic acid of claim 1 wherein the nucleic acid is NA10.1 and L10₂ represents A.

25. The nucleic acid of claim 1 wherein the nucleic acid is NA10.1 and L10₃ represents CGT or CGU.

26. The nucleic acid of claim 1 wherein the nucleic acid is NA10.1 and L10₄ represents C.

27. The nucleic acid of claim 1 wherein the nucleic acid is NA10.1 and is represented by SEQ ID NO:2 or SEQ ID NO:3.

28. (canceled)

29. The nucleic acid of claim 1 wherein the nucleic acid is NA10.2 and L10₁ represents A.

30. The nucleic acid of claim 1 wherein the nucleic acid is NA10.2 and L10₂ represents A.

31. The nucleic acid of claim 1 wherein the nucleic acid is NA10.2 and L10₃ represents CGT or CGU.

32. The nucleic acid of claim 1 wherein the nucleic acid is NA10.2 and L10₄ represents CCT or CCU.

33. The nucleic acid of claim 1 wherein the nucleic acid is NA10.2 and L10₅ represents C.

34. The nucleic acid of claim 1 wherein the nucleic acid is NA10.2 and is represented by SEQ ID NO:4 or SEQ ID NO:5.

35. (canceled)

36. The nucleic acid of claim 1 wherein the nucleic acid is NA10.3 and L10₃ represents T or U.

37. The nucleic acid of claim 1 wherein the nucleic acid is NA10.3 and L10₄ represents CCT or CCU.

38. The nucleic acid of claim 1 wherein the nucleic acid is NA10.3 and L10₅ represents C.

39. The nucleic acid of claim 1 wherein the nucleic acid is NA10.3 and L10₆ represents ACT or ACU.

40. The nucleic acid of claim 1 wherein the nucleic acid is NA10.3 and is represented by SEQ ID NO:6 or SEQ ID NO:7.

41. (canceled)

42. The nucleic acid of claim 1 wherein the nucleic acid is NA10.4 and L10₄ represents CCT or CCU.

43. The nucleic acid of claim 1 wherein the nucleic acid is NA10.4 and L10₅ represents C.

44. The nucleic acid of claim 1 wherein the nucleic acid is NA10.4 and L10₆ represents ACT or ACU.

45. The nucleic acid of claim 1 wherein the nucleic acid is NA10.4 and L10₇ represents AGT or AGU.

46. The nucleic acid of claim 1 wherein the nucleic acid is NA10.4 and is represented by SEQ ID NO:8 or SEQ ID NO:9.

47. A method of treating a lentivirus infection in a subject in need thereof comprising administering to the subject a therapeutically effective amount of at least one nucleic acid selected from the group consisting of
i) a nucleic acid (NA3) having the general formula (I) of:
L3₁-(G)₄₋₆-L3₂-(G)₄₋₆-L3₃-(G)₃₋₄-L3₄ (I)
wherein
L3₁ represents a sequence of 0 to 4 nucleotides
L3₂ represents a sequence of 2 to 5 nucleotides
L3₃ represents a sequence of 5 to 10 nucleotides and
L3₄ represents a sequence of 0 to 4 nucleotides;
ii) a nucleic acid (NA9) having the general formula (II) of:
L9₁-(G)₄₋₆-L9₂-(G)₂₋₃-L9₃-(G)₂₋₄-L9₄ (II)
wherein
L9₁ represents a sequence of 0 to 4 nucleotides
L9₂ represents a sequence of 2 to 4 nucleotides
L9₃ represents a sequence of 1 to 4 nucleotides and
L9₄ represents a sequence of 0 to 4 nucleotides;
iii) a nucleic acid (NA10) having the general formula (III) of:
L10₀-(G)₃₋₄-L10₁-(G)₃₋₄-L10₂-(G)₂₋₃-L10₃-(G)₂₋₃-L10₄-(G)₃₋₄-L10₅-(G)₃₋₄-L10₆-(G)₃₋₄-L10₇-(G)₂₋₃-L10₈ (III)
wherein
L10₀ represents a sequence of 0 to 4 nucleotides
L10₁ represents a sequence of 7 to 10 nucleotides
L10₂ represents a sequence 1 to 3 nucleotides
L10₃ represents a sequence of 1 to 4 nucleotides
L10₄ represents a sequence 2 to 4 nucleotides
L10₅ represents a single nucleotide
L10₆ represents a sequence of 2 to 5 nucleotides
L10₇ represents a sequence of 2 to 5 nucleotides and
L10₈ represents a sequence of 0 to 4 nucleotides;
iv) a nucleic acid (NA10.1) having the general formula (IV) of:
L10₀-(G)₃₋₄-L10₁-(G)₃₋₄-L10₂-(G)₂₋₃-L10₃-(G)₂₋₃-L10₄ (IV)
wherein
L10₀ represents a sequence of 0 to 4 nucleotides
L10₁ represents a sequence of 7 to 10 nucleotides
L10₂ represents a sequence of 1 to 3 nucleotides
L10₃ represents a sequence of 1 to 4 nucleotides and
L10₄ represents a sequence of 0 to 4 nucleotides;
v) a nucleic acid (NA10.2) having the general formula (V) of:
L10₁-(G)₃₋₄-L10₂-(G)₂₋₃-L10₃-(G)₂₋₃-L10₄-(G)₃₋₄-L10₅-(G)₃₋₄-L10₆ (V)
wherein
L10₁ represents a sequence of 0 to 4 nucleotides
L10₂ represents a sequence of 1 to 3 nucleotides
L10₃ represents a sequence of 1 to 4 nucleotides
L10₄ represents a sequence of 2 to 4 nucleotides
L10₅ represents a single nucleotide and
L10₆ represents a sequence of 0 to 4 nucleotides;
vi) a nucleic acid (NA10.3) having the general formula (VI) of:
L10₃-(G)₂₋₃-L10₄-(G)₃₋₄-L10₅-(G)₃₋₄-L10₆-(G)₃₋₄-L10₇ (VI)
wherein
L10₃ represents a sequence of 0 to 4 nucleotides
L10₄ represents a sequence 2 to 4 nucleotides
L10₅ represents a single nucleotide and
L10₆ represents a sequence of 2 to 5 nucleotides
L10₇ represents a sequence of 0 to 4 nucleotides; and
vii) a nucleic acid (NA10.4) having the general formula (VII) of:
L10₄-(G)₃₋₄-L10₅-(G)₃₋₄-L10₆-(G)₃₋₄-L10₇-(G)₂₋₃-L10₈ (VII)
wherein
L10₄ represents a sequence of 0 to 4 nucleotides
L10₅ represents a single nucleotide
L10₆ represents a sequence of 2 to 5 nucleotides
L10₇ represents a sequence of 2 to 5 nucleotides and
L10₈ represents a sequence of 0 to 4 nucleotides;
and wherein for each of NA3, NA9, NA10, NA10.1, NA10.3, NA10.2, and NA10.3, (G)₂₋₃, (G)₃₋₄ and (G)₄₋₆ represent a sequence of 2 to 3, 3 to 4 or 4 to 6 guanosines, respectively.

48. (canceled)

49. A pharmaceutical composition comprising one or more nucleic acids selected from the group consisting of
i) a nucleic acid (NA3) having the general formula (I) of:
L3₁-(G)₄₋₆-L3₂-(G)₄₋₆-L3₃-(G)₃₋₄-L3₄ (I)
wherein
L3₁ represents a sequence of 0 to 4 nucleotides
L3₂ represents a sequence of 2 to 5 nucleotides
L3₃ represents a sequence of 5 to 10 nucleotides and
L3₄ represents a sequence of 0 to 4 nucleotides;
ii) a nucleic acid (NA9) having the general formula (II) of:
L9₁-(G)₄₋₆-L9₂-(G)₂₋₃-L9₃-(G)₂₋₄-L9₄ (II)
wherein
L9₁ represents a sequence of 0 to 4 nucleotides
L9₂ represents a sequence of 2 to 4 nucleotides
L9₃ represents a sequence of 1 to 4 nucleotides and
L9₄ represents a sequence of 0 to 4 nucleotides;
iii) a nucleic acid (NA10) having the general formula (III) of:
L10₀-(G)₃₋₄-L10₁-(G)₃₋₄-L10₂-(G)₂₋₃-L10₃-(G)₂₋₃-L10₄-(G)₃₋₄-L10₅-(G)₃₋₄-L10₆-(G)₃₋₄-L10₇-(G)₂₋₃-L10₈ (III)
wherein
L10₀ represents a sequence of 0 to 4 nucleotides
L10₁ represents a sequence of 7 to 10 nucleotides
L10₂ represents a sequence 1 to 3 nucleotides
L10₃ represents a sequence of 1 to 4 nucleotides
L10₄ represents a sequence 2 to 4 nucleotides
L10₅ represents a single nucleotide
L10₆ represents a sequence of 2 to 5 nucleotides
L10₇ represents a sequence of 2 to 5 nucleotides and
L10₈ represents a sequence of 0 to 4 nucleotides;
iv) a nucleic acid (NA10.1) having the general formula (IV) of:
L10₀-(G)₃₋₄-L10₁-(G)₃₋₄-L10₂-(G)₂₋₃-L10₃-(G)₂₋₃-L10₄ (IV)
wherein
L10₀ represents a sequence of 0 to 4 nucleotides
L10₁ represents a sequence of 7 to 10 nucleotides
L10₂ represents a sequence of 1 to 3 nucleotides
L10₃ represents a sequence of 1 to 4 nucleotides and
L10₄ represents a sequence of 0 to 4 nucleotides;
v) a nucleic acid (NA10.2) having the general formula (V) of:
L10₁-(G)₃₋₄-L10₂-(G)₂₋₃-L10₃-(G)₂₋₃-L10₄-(G)₃₋₄-L10₅-(G)₃₋₄-L10₆ (V)
wherein
L10₁ represents a sequence of 0 to 4 nucleotides
L10₂ represents a sequence of 1 to 3 nucleotides
L10₃ represents a sequence of 1 to 4 nucleotides
L10₄ represents a sequence of 2 to 4 nucleotides
L10₅ represents a single nucleotide and
L10₆ represents a sequence of 0 to 4 nucleotides;
vi) a nucleic acid (NA10.3) having the general formula (VI) of:
L10₃-(G)₂₋₃-L10₄-(G)₃₋₄-L10₅-(G)₃₋₄-L10₆-(G)₃₋₄-L10₇ (VI)
wherein
L10₃ represents a sequence of 0 to 4 nucleotides
L10₄ represents a sequence 2 to 4 nucleotides
L10₅ represents a single nucleotide and
L10₆ represents a sequence of 2 to 5 nucleotides
L10₇ represents a sequence of 0 to 4 nucleotides; and
vii) a nucleic acid (NA10.4) having the general formula (VII) of:
L10₄-(G)₃₋₄-L10₅-(G)₃₋₄-L10₆-(G)₃₋₄-L10₇-(G)₂₋₃-L10₈ (VII)
wherein
L10₄ represents a sequence of 0 to 4 nucleotides
L10₅ represents a single nucleotide
L10₆ represents a sequence of 2 to 5 nucleotides
L10₇ represents a sequence of 2 to 5 nucleotides and
L10₈ represents a sequence of 0 to 4 nucleotides;
and wherein for each of NA3, NA9, NA10, NA10.1, NA10.3, NA10.2, and NA10.3, (G)₂₋₃ (G)₃₋₄ and (G)₄₋₆ represent a sequence of 2 to 3, 3 to 4 or 4 to 6 guanosines, respectively.
